(19) United States
(12) Patent Application Publication
(10) Pub. No.: US 2012/0184921 A1
(43) Pub. Date: Jul. 19, 2012

(54) CANNULA FOR A LARYNGOTRACHEAL ANESTHESIA DEVICE

(76) Inventor: Vianney Brillant, Iguerande (FR)

(21) Appl. No.: 13/395,693

(22) PCT Filed: Oct. 1, 2010
(86) PCT No.: PCT/FR10/00654

§ 371 Date: Mar. 13, 2012

(51) Int. Cl. A61M 19/00; A61M 25/16

(52) U.S. Cl. 604/239; 604/533

(57) ABSTRACT

The invention relates to a cannula (5) for a laryngotracheal anesthesia device (1) comprising a semi-rigid elongate tube (6) including a proximal end (14), to be connected to a syringe (2) containing the anesthetic liquid, and a distal end (15), to be inserted into the cavity of the larynx up until it is inside the trachea of a patient, a plurality of anesthetic liquid outlets (16) being provided, from the distal end (15), on a predetermined area of the length of said tube (6). According to the invention, said tube (6) is inwardly curved over the entire length thereof between said proximal end (14) and said distal end (15), and said tube (6) is open at the distal end (15) thereof and makes it possible to receive an assembly forming a flexible tip (9) comprising an axial outlet (33) for liquid anesthetic.

### CROSS-REFERENCE TO RELATED APPLICATIONS

The present application is a National Stage Application of PCT International Application No. PCT/FR2010/000654 (filed on Oct. 1, 2010), under 35 U.S.C. §371, which claims priority to French Patent Application No. 0904699 (filed on Oct. 2, 2009), which are each hereby incorporated by reference in their respective entireties.

### FIELD OF THE INVENTION

The invention relates to a cannula for a laryngotracheal anesthesia device and an anesthesia device having a cannula.

### BACKGROUND OF THE INVENTION

A cannula is a device intended to be used for anesthesia of the tracheal region before certain medical interventions, in particular in order to suppress the tracheal cough reflex. Indeed, the recurrent vagus nerve or inferior laryngeal nerve ensures the sensory innervation of the infraglottic portion of the larynx and the trachea. It also provides the intrinsic muscles of the larynx with motor contingency. The external branch of the superior laryngeal nerve innervates the sensitivity of the anterior portion of the infraglottic mucous membrane of the larynx, and provides the cricothyroid muscle, or the tensor muscle of the vocal cords, with exclusive motor contingency. Thus, a sensory block of the entire respiratory tract must be produced by blocking the superior laryngeal nerve, in association with anesthesia in contact with the infraglottic and tracheal mucous membranes.

Local intratracheal anesthesia is performed by local installation of liquid. anesthetic by insertion of a multi-perforated stylus positioned in the tracheal system beyond the vocal cords.

Such a device used to produce local intratracheal anesthesia is described in the document U.S. Pat. No. 4,182,326. This document describes a drug injector for laryngotracheal anesthesia including an ampoule containing the liquid drug intended to be installed inside a syringe equipped at its discharge end-piece with a long cannula comprising fluid discharge openings. The cannula is flexible but semi-rigid, has a rectilinear shape over a large portion of its length, in particular in its upper portion connecting it to the syringe, and is curved over its lower portion. The curved portion of the cannula includes a plurality of discharge openings for anesthetic liquid arranged in a staggered formation. When the anesthetic liquid is pushed into the cannula, the lower half thereof produces spiral jets that spray the interior of the trachea. While its operation is satisfactory, it has been noted that the shape of the cannula of the device was not adapted to the anatomical shape of the passage through which it passes when it is inserted into the trachea. In fact, when it is handled by the medical staff, the semi-rigid end-piece is, indeed, easy to insert, owing to the curved shape of its leading portion, into the passage, but it conforms thereto only because of its deformability, when it is handled by the medical staff. Therefore, the medical staff must be well trained in order to insert t into the larynx, in particular since such an operation is generally performed blindly. Moreover, the semi-rigid end-piece of the cannula may run into the vocal cords located at the entrance of the laryngeal cavity. Furthermore, the spiral shape of the drug jet discharged through the openings of the cannula and the lack of a liquid discharge opening at the end of the cannula do not enable effective spraying of the area to be anesthetized.

In addition, U.S. Design Pat. No. D245,120 discloses a laryngotracheal device similar to that of the above-described document, but in which the cannula includes a curved portion extending over a longer length than that of the above-described document. However, the cannula of this document includes discharge openings that are located only on its side, resulting in a non-uniform distribution of the anesthetic product inside the trachea. Moreover, the discharge end of the cannula may injure the tissue of the passages through which it passes as it is inserted into the throat of a patient.

Another type of cannula used to provide laryngotracheal anesthesia is also known from U.S. Pat. No. 4,402,684. According to this document, the cannula has an elongate shape, it is curved at its free end, and its opposite end is connected to a syringe containing the anesthetic product. The cannula includes a rigid internal rod having an X-shaped cross-section covered over its entire length with a flexible tube. The anesthetic liquid passes through the longitudinal channels of the cannula, with the channels being formed between the rod and the surrounding tube, and is discharged through the side and end openings of the latter. In spite of the fact that the external tube has a flexible end-piece giving it greater flexibility in contact with the mucous membranes of the passages through which it passes, it is noted that the rectilinear shape that the cannula has over almost its entire length is not at all adapted to the anatomical shape of the passage connecting the laryngeal cavity to that of the trachea. Moreover, the rigid internal rod does not enable it to be adapted to the various shapes and sizes of the conduits of each individual.

An improved solution was described in the document WO 02/09799, in which the cannula of the laryngotracheal anesthesia device includes a tube reinforced with a stiffener made of a malleable material, in which the tube includes a flexible curved end that is equipped with anesthetic product distribution openings. Such a cannula can be inserted, then oriented in the laryngotracheal passage, by causing it to turn therein, without injuring the wall tissue. It is noted, however, that the arrangement of the distribution openings does not enable uniform distribution of the anesthetic product in the trachea. Moreover, the insertion of the cannula into the trachea requires additional orientation and rotation operations that lengthen the handling time, all while risking irritating the walls of the passages with which it comes into contact.

### SUMMARY OF THE INVENTION

The objective of the invention is to overcome at least some of these disadvantages and to propose a cannula for a laryngotracheal anesthesia device, in particular before performing endotracheal intubation, which cannula is better adapted to the anatomy of the aerodigestive tract (the term aerodigestive tract refers to a passage that begins in the oral cavity and leads to the interior of the trachea) of different patients, which is capable of protecting the vocal cords of the patients as it is inserted into the trachea via the laryngeal cavity, while enabling fast and effective anesthesia of the laryngotracheal passage to be provided.

Another objective of the invention is to provide a cannula for a laryngotracheal anesthesia device that is ergonomic and easy for the medical staff to handle, without presenting the slightest risk to the patient, while enabling uniform distribution of the anesthetic product to be provided in the laryngotracheal passage and thus effective anesthesia thereof.

Another objective of the invention is to provide a cannula for a laryngotracheal anesthesia device with effective and reliable operation, which also has a simple design and is inexpensive to produce in mass production.

These objectives are achieved by a cannula for a laryngotracheal anesthesia device comprising a semi-rigid elongate tube including a proximal end intended to be connected to a syringe containing the anesthetic liquid and a distal end intended to be inserted into the laryngeal cavity to the trachea of a patient, in which a plurality of anesthetic liquid discharge openings are provided starting at the distal end, over a predetermined area along the length of the tube, because the tube is curved over its entire length between the proximal end and the tube is open at its distal end and makes it possible to receive an assembly forming a flexible end-piece comprising an axial anesthetic liquid discharge opening.

The cannula in accordance with the invention is intended to be used with a syringe containing the anesthetic liquid by connecting it to the discharge end-piece of the latter, for example by using a Luer-type joint. The cannula includes a semi-rigid cylindrical tube having two ends: a proximal end for attachment to the syringe and a distal end for insertion into the oral, then laryngeal cavity, and up to the trachea of a patient. In accordance with the invention, the cannula includes a tube that has a curved shape over its entire length. The curved shape is chosen so that it conforms or is similar to the anatomical shape of the aerodigestive tract formed by the oral cavity, the pharynx, the laryngeal cavity and the trachea.

Such a curved shape already enables it to naturally follow the arched shape of the oral cavity, then the pharyngeal passage, as well as the epiglottis passage and the entrance to the laryngeal cavity, as well as the entrance to the trachea. Thus, as seen in the axial cross-section produced along a vertical mid-plane, the common axis passing through the axes of all of these passages has a general arch shape, which is substantially that of an are of circle.

The cannula of the invention therefore includes a semi-rigid tube of which the distal end is open and makes it possible to receive an assembly forming a flexible end equipped with an axial opening (that is, an opening produced along the longitudinal axis of the tube). The tube of the cannula of the invention is semi-rigid, it has a shape and size conforming to the anatomical shape and size of the human body. This semi-rigid tube is completed by an assembly forming an end-piece of which the portion intended to come into contact with the internal passage walls of the human body is made of a different material that is more flexible than that of the tube.

A cannula thus obtained has good properties of orientation and adaptation to the passages of different patients, owing to the curved shape and the elasticity properties of the semi-rigid tube, as well as the fast and easy insertion owing to the presence of the flexible end-piece that can be moved without the risk of injuring tissue. Thus, a fast insertion of the cannula is achieved, which then makes it possible to distribute the anesthetic product uniformly and over a larger area of the trachea, and therefore enables fast and effective anesthesia of the treated passage to be provided,

It is also easier to pass the vocal cords with the cannula of the invention, as the cannula is naturally positioned, during its insertion, owing to its ergonomic shape adapted to the shape of the passages through which it passes, without running into the walls of the passages or injuring the tissue of the latter. In addition, the flexible end-piece is provided with an axial anesthetic product opening, which makes it possible to send the anesthetic liquid even further inside the trachea. Such a flexible end-piece makes it possible to protect the mucous membranes and prevents any risk of damage to the vocal cords when any contact is made with them. This is very useful for blind insertion of the cannula into the laryngotracheal cavity of a patient,

Thus, owing to its curved shape over its entire length, the cannula conforms to the aerodigestive tract through which it passes, which means that it is easy to insert, ergonomic and safe for the medical staff, while enabling effective anesthesia of the passage to be provided. In addition, owing to its flexibility, it can be easily adapted to any morphological variations that may exist between individuals. Moreover, such a cannula can be produced inexpensively, by using an open tube of which one of the ends is closed by adding an end-piece that can, for example, be force-fitted into the tube.

Preferably, the assembly forming the end-piece includes a flexible sleeve that covers a rigid nozzle inserted at the distal end of the tube. Such a rigid nozzle has a calibrated discharge opening, with a predetermined shape and size, enabling effective atomization of the fluid jet discharged. The nozzle is covered with a flexible sleeve for protection of the assembly forming the end-piece. This makes it possible to obtain a small assembly forming an end-piece, having properties of flexibility in contact with the tissue, while providing an atomized discharge jet.

In addition, the design of the cannula is simple and inexpensive to produce, from a cylindrical tube open at its ends, with the distal end being capable of then being closed by a nozzle, equipped with an axial or end discharge opening, and covered with a flexible sleeve including an axial opening in the extension of the axial nozzle discharge opening.

Advantageously, the nozzle has an axial discharge opening with a diameter smaller than that of the internal diameter of the tube. This makes it possible to increase the jet discharge speed, enabling the liquid to be distributed quickly, even at the base of the trachea, for a better anesthetic effect.

Preferably, the nozzle includes an internal conduit including at least two coaxial cylindrical conduit segments of which the diameter decreases in the direction of the axial discharge opening.

Such a design of the internal conduit of the nozzle enables the anesthetic product jet to be accelerated and better atomization of the product discharged from the nozzle to be obtained.

Preferably, the sleeve has an axial opening with a generally flared conical shape, and it is coaxial with the axial discharge opening of the nozzle. This enables the anesthetic fluid to be expanded into a cone-shaped jet, over a larger area, for more effectiveness on the tissue of the laryngotracheal passage. Advantageously, the assembly forming a flexible end-piece is produced by a resilient snap-fastening of the flexible sleeve on the nozzle, and the assembly is attached by force-fitting the nozzle inside the tube.

Such a production makes it possible to obtain a solid mechanical assembly, while avoiding the use of any glue or adhesive capable of causing harm to the body. Thus, by carefully choosing the materials of the assembly parts, good biocompatibility thereof is obtained.

Advantageously, the external diameter of the flexible end-piece is substantially equal to the diameter of the tube. This makes it possible to obtain a cannula with a smaller diameter, enabling easy insertion, even in the narrow passages of the aerodigestive tract (such as the epiglottis), and without the risk of injuring the tissue of the passage.

Preferably, in accordance with the invention, the tube has an arc of circle shape with a radius of between 140 mm and 190 mm, and a length of between 170 mm and 225 mm, in accordance with the morphology and/or the age of the patient,

Thus, in tests performed with a plurality of curved cannula, it has been noted that an arc of circle shape with a radius and length established in accordance with the invention is best adapted to the human anatomy.

Preferably, the discharge openings are produced in the form of a plurality of parallel rows with radial openings, in which the axes of the openings of one row are coplanar and uniformly distributed over the circumference of the tube.

This makes it possible to obtain a uniform spray, over the entire circumference of the laryngotracheal cavity, with a very good anesthetic effect.

Advantageously, the cannula in accordance with the invention includes at least six parallel rows of discharge openings, preferably eight parallel rows of discharge openings, in which each row has four discharge openings and extends over a length substantially equal to or greater than half the length of the tube.

It has been established in laboratory tests that such a ratio of number of openings over the length of the cannula ensured a good distribution of anesthetic liquid over the treated area of the laryngotracheal cavity,

Preferably, the tube includes two markings printed or etched on the external surface of the tube. These markings form visual reference points informing the person handling the anesthesia device of the arrival, then the passing of the vocal cords, so that the insertion of the cannula is performed more carefully and even more slowly.

The objectives of the invention are also achieved with a laryngotracheal anesthesia device including a cannula in accordance with the invention.

Preferably, the anesthesia device of the invention includes a syringe intended to contain the anesthetic liquid, a needle intended to pump the anesthetic liquid into the syringe and the cannula equipped with the end-piece for connection to the syringe, with the entire assembly being contained inside a sterile packaging. This makes it possible to have all of the components of the device in a sterile kit that is easy to use and ready for use in a hospital setting.

### BRIEF DESCRIPTION OF THE DRAWINGS

FIG. 1 illustrates an exploded overview of the anesthesia device in accordance with the invention.

FIG. 2 illustrates a longitudinal cross-section view of a cannula in accordance with the invention, in which the proximal end thereof is connected to a connection end-piece for connection to the syringe of the device.

FIG. 3 illustrates a longitudinal cross-section view, on an enlarged scale, of an assembly firming a flexible end-piece of the cannula of the invention.

FIGS. 4a and 4b illustrate respectively a perspective view, on an enlarged scale, of a nozzle of the flexible assembly of the cannula of the invention, and a longitudinal cross-section view of the nozzle of FIG. 4a.

FIGS. 5a and 5b illustrate respectively a longitudinal cross-section view of a cannula in accordance with the invention, and a transverse cross-section view, on an enlarged scale, produced with a cross-section plane perpendicular to the longitudinal axis of the cannula and passing through the center of the discharge openings.

### LIST OF REFERENCES

| | |
|---|---|
| 1 | Laryngotracheal anesthesia device |
| 2 | Syringe |
| 3 | Piston |
| 4 | Connection end-piece |
| 5 | Cannula |
| 6 | Tube |
| 7 | Syringe body |
| 9 | Assembly forming flexible end-piece |
| 10 | Front portion of syringe |
| 11 | Threaded portion |
| 12 | Front end |
| 13 | Conduit |
| 14 | Proximal end |
| 15 | Distal end |
| 16 | Discharge openings |
| 17 | Lower reference point |
| 18 | Upper reference point |
| 19 | Longitudinal axis of tube |
| 20 | Flexible sleeve |
| 21 | Rigid nozzle |
| 22 | Body |
| 23 | Rear portion |
| 24 | Groove |
| 25 | Front portion |
| 26 | Guide portion |
| 27 | Flexible lip |
| 28 | Leading portion |
| 29 | Inlet channel |
| 30 | Intermediate channel |
| 31 | Discharge channel |
| 32 | Axial opening |
| 33 | Axial discharge |
| 34 | Edge |

### DETAILED DESCRIPTION OF EMBODIMENTS

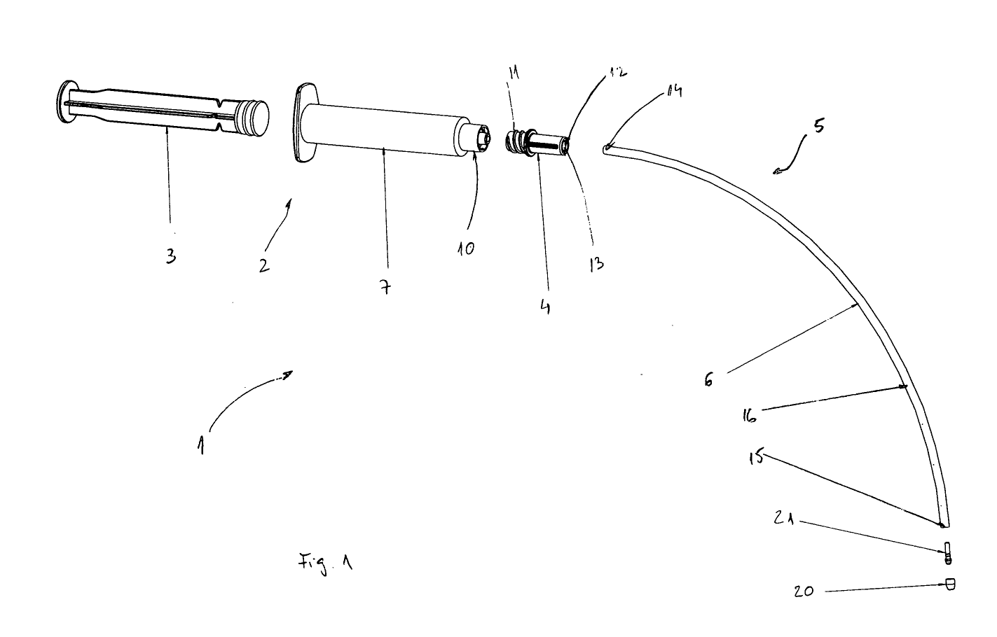

FIG. 1 illustrates an example of an embodiment of a laryngotracheal anesthesia device 1 in accordance with the invention, including a syringe 2 intended to contain an anesthetic liquid and a cannula 5 that is attached to the syringe 2 by means of a connection end-piece 4, in particular, of the Luer joint type. The syringe 2 includes a cylindrical syringe body 7 intended to contain the anesthetic liquid, such as, for example, lidocaine hydrochloride. The anesthetic liquid is confined inside a sealed flask. The device 1 also includes a needle (not shown in the drawings) that is attached to the syringe 2 so as to pump the anesthetic liquid from the flask into the syringe 2. The rear portion of the syringe 2 receives a piston 3 that cooperates with the internal cylindrical portion of the syringe body 7, enabling, when actuated, the anesthetic liquid to be pushed toward the front portion 10 of the syringe 2. The front portion 10 of the syringe is equipped with an internal threading that cooperates with the threaded portion 11 of the connection end-piece 4, which is, in this example, a Luer joint generally known to a person skilled in the art.

The connection end-piece 4 is a part with a general tubular shape, with its front end 12 including a conduit 13 into which the proximal end 14 of the tube 6 is inserted by a force-fitting assembly, in which the end-piece 4 and the tube 6 form part of the cannula assembly 5. The connection end-piece 4 thus enables a removable attachment of the cannula 5 to the syringe 2. Such a connection end-piece 4 is preferably made of a plastic material, such as polypropylene.

The cannula 5 includes a semi-rigid elongate tube 6 having an open proximal end 14 inserted into the connection end-piece 4 and a distal end 15 that, in the example illustrated, is also open. Anesthetic liquid discharge openings 16 are also provided from the distal end 15, over around half the length of the tube, as will be explained below. More specifically, in accordance with the invention, the tube 6 has a curved shape over its entire length between the proximal end and the distal end so as to enable it to be better adapted to the human anatomy,

In the example illustrated in the figures, the tube 6 is a semi-rigid cylindrical tube, preferably transparent. Such a transparent tube 6 makes it possible to visually see the advancement of the anesthetic liquid from the syringe to the vicinity of the first discharge opening 16 before the cannula 5 is inserted into the patient's oral cavity.

The tube 6 is made of a plastic material by an extrusion technique. Such a plastic material can be polypropylene or polyethylene or any other plastic material having similar properties, with the exception of polyvinyl chloride. The tube 6, which is initially rectilinear, is heated to a temperature of around 80° C., then deformed on a matrix or a template, which ensures that it is shaped in accordance with the desired curvature. As an example, the tube 6 has a length of between 170 mm and 225 mm, preferably equal to 205 mm and an arc of circle shape with a radius of between 140 mm and 190 mm and preferably equal to 173 mm. In the example illustrated, the tube 6 has an external diameter of around 3 mm and an internal diameter of around 2.2 mm.

The tube 6 can be seen better in FIG. 5a. A plurality of discharge openings 16 distributed in rows are drilled in the thickness of the tube 6. In accordance with an advantageous embodiment of the invention, the discharge openings 16 are produced in the form of a plurality of parallel rows with radial openings. The axes of the openings of one row are coplanar and uniformly distributed over the circumference of the tube 6. In the example illustrated in the figures, the tube 6 includes eight parallel rows of discharge openings 16, in which each row has four discharge openings 16 arranged at 90°, as is better illustrated in FIG. 5b. The rows of discharge openings 16 are uniformly distributed over the length of the tube 6 and extend, from the distal end 15 of the tube 6, over a length substantially equal to or greater than half the length of the latter. In the example illustrated, the openings 16 have a diameter of 0.1 mm and are produced by vacuum drilling through the thickness of the tube 6, and the openings 16 are produced after the shaping by heating of the tube 6.

In accordance with another advantageous aspect of the invention, the tube 6 includes two markings printed or etched on the external surface of the tube. These markings are visual reference points informing the person handling the anesthesia device of the arrival, then the passing of the vocal cords, so that the insertion of the cannula 5 is performed more carefully and even more slowly. Thus, a first marking forms a lower reference point 17, which indicates the arrival of the distal end of the cannula 5 in the area of the vocal cords, and the second marking forms an upper reference point 18, which indicates that this area has been passed. The openings 16 extend from the lower reference point 17 to the vicinity of the distal end 15. The eight rows of the discharge openings 16 are uniformly distributed over the aforementioned length of the tube 6, with their axes extending perpendicularly to the longitudinal axis 19 of the tube 16, in particular in accordance with radii extending from the center O of curvature of the tube 6 (FIG. 5a).

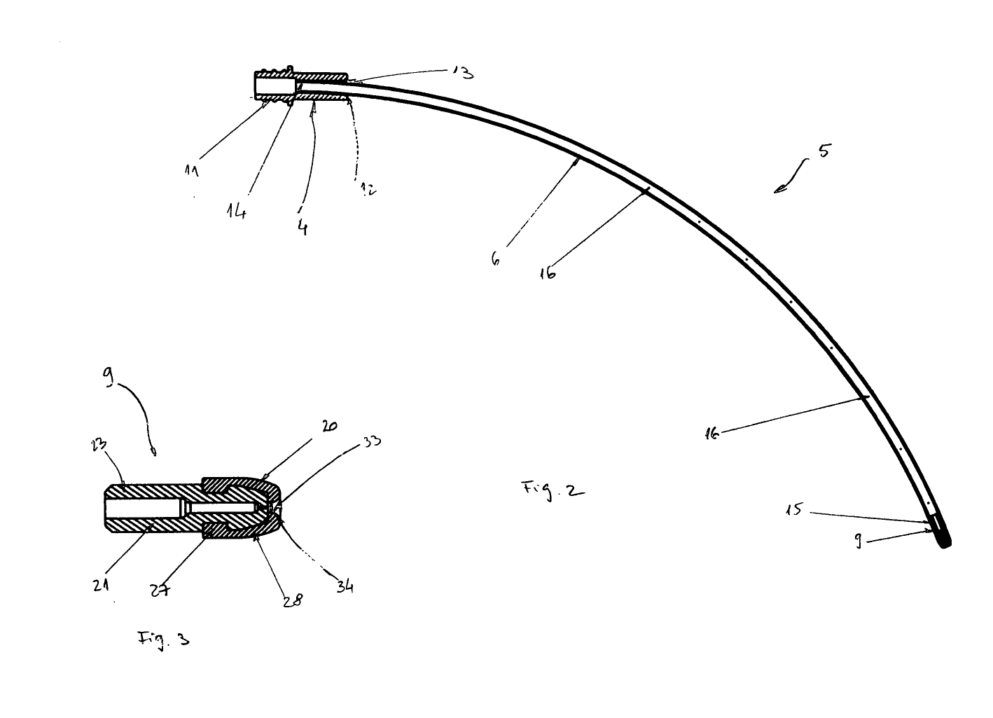

In accordance with yet another embodiment of the invention, which can be combined with those described above, the cannula 5 is equipped with an assembly forming a flexible end-piece 9 at its distal end 15. Such an assembly 9 can be seen better in FIG. 3 and includes a flexible sleeve 20, which covers a rigid nozzle 21.

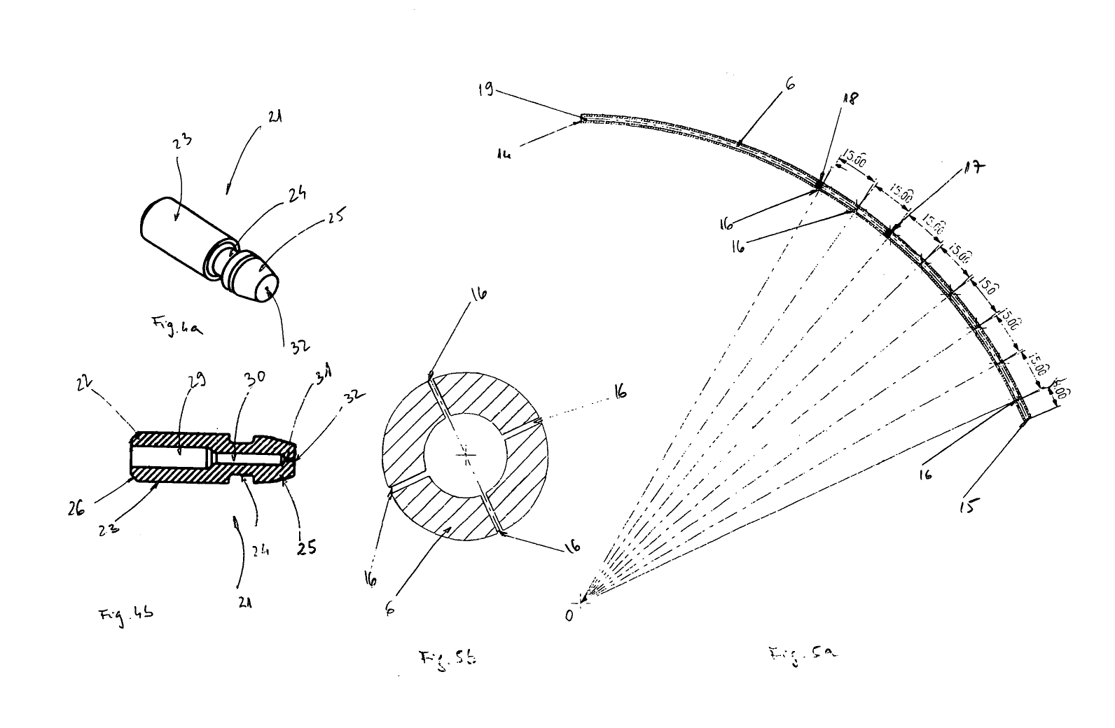

The nozzle 21 can be seen better in FIGS. 4a and 4b. It includes a body 22 having a rotary shape having three portions: a cylindrical rear portion 23, a cylindrical groove 24 and a front portion 25 with a general conical shape. The rear portion 23 has an external diameter similar to that inside the tube 6, and is intended to be inserted by force-fitting inside the tube 6. The rear portion 23, therefore, includes a chamfered guide portion 26. The groove 24 is used for attachment by resilient snap-fastening with the flexible sleeve 20, which sleeve therefore, includes a flexible lip 27 (FIG. 3). The conical shape of the front portion 25 is covered by the leading portion 28 of the flexible sleeve 20 (FIG. 3). The leading portion 28 matches the shape of the front portion 25 of the nozzle 21 and enables the insertion of the distal end 15 of the cannula into the patient's throat to be facilitated.

In the example illustrated, the body 22 of the nozzle 21 has an external diameter of 2.36 mm and a total length of 8 mm. As can be seen better in FIG. 4b, the body 22 of the nozzle 21 is internally passed through by a channel with three segments having diameters that decrease from the rear portion 23: a first segment forming an inlet channel 29 with a diameter of around 1 mm and a length of around 4.2 mm, a second segment forming an intermediate channel 30 with a diameter of around 0.5 mm and a length of around 3.3 mm, and a third segment forming a discharge channel 31 with a diameter of around 0.1 mm and a length of around 0.5 mm. The nozzle 21 receives the anesthetic liquid coming from the tube 6, and the decreasing diameters of the segments of the channels passing through it enable the liquid jet to be accelerated progressively before it is discharged through an axial opening 32 thereof The configuration of the discharge channel 31 as well as that of the axial opening 32 are produced so as to enable atomization of the anesthetic liquid jet discharged through the end-piece 9 of the cannula 5.

In reference to FIG. 3, it is noted that the flexible sleeve 20 has a flared conical opening or axial discharge 33 that is coaxial with the axial opening 32 of the nozzle 21, and therefore, with the longitudinal axis of the tube 6. The opening or axial discharge 33 has a flared shape having rounded edges 34, enabling the anesthetic liquid discharged through the axial opening 32 to be sprayed over a larger area. As an example, the sleeve 20 has an external diameter of around 3 mm and a length of around 41 mm, with the diameter of the front end of the leading portion 28 being around 2 mm. The flexible sleeve 20 is preferably made of a flexible plastic material by an injection technique. Such a material can, for example, be a polyolefin-based thermoplastic material, such as Santoprerie TPV™, sold by ExxonMobil, having a Shore A hardness equal to 62. In another alternative, it can also be made of medical silicone.

The assembly forming a flexible end-piece 9 is produced by snap-fastening the sleeve 20 on the nozzle 21. Then, the assembly is positioned by force-fitting the rear portion 23 of the nozzle 21 inside the tube 6. Thus, it is noted that the assembly forming a flexible end-piece 9 does not exceed the external diameter of the tube 6 and extends over a length equal to that of the flexible sleeve 20 from the distal end 15 of the tube 6. Therefore, the cannula 5 having the assembly 9 is easy to introduce into the laryngotracheal cavity of the patient and has a flexible end that prevents any risk of injury to the vocal cords or mucous membranes of the passages through which it passes.

During use of the anesthesia device 1, in particular before performing endotracheal intubation during certain surgical interventions, the cannula 5 is inserted through the oral cavity of the patient. Once the cannula is in place, the piston 3 is actuated, which enables the anesthetic liquid to be sent into the patient's laryngotracheal cavity. The cannula 5 is then removed and the patient is intubated.

Other alternatives and embodiments of the invention can be produced without going beyond the scope of the claims.

Thus, in one alternative, the cannula can be removably attached to the syringe 2, for example, by directly and securely coupling the proximal end 14 of the tube 6 to the discharge end of the syringe 2. In another alternative, the flexible sleeve 20 is attached by force-fitting onto the rigid nozzle 21.

1-13. (canceled)

14. A cannula comprising:
a tube which is curved over an entire length thereof;
a plurality of tube discharge openings provided on the tube;
an end piece configured for receipt at a distal end of the tube and having a discharge opening.

15. The cannula of claim 14, wherein the tube comprises a semi-rigid elongate tube.

16. The cannula of claim 14, wherein the tube discharge openings are provided along the length of the tube between the distal end of the tube and a predetermined area of the tube.

17. The cannula of claim 14, wherein the end-piece comprises:
a nozzle configured for receipt at the distal end of the tube; and
a sleeve that covers the nozzle, the sleeve having an axial discharge.

18. The cannula of claim 17, wherein the nozzle has an axial discharge opening with a diameter less than the internal diameter of the tube.

19. The cannula of claim 18, wherein the nozzle has an internal conduit comprising at least two internal conduit segments which are coaxial relative to each other.

20. The cannula of chin 19, wherein the diameter of the at least two internal conduits decreases in the direction of the axial opening.

21. The cannula of claim 18, wherein the axial discharge of the sleeve has a flared conical shape, the axial discharge being coaxial with the axial discharge opening of the nozzle.

22. The cannula of claim 14, wherein:
the sleeve is snap-fasten to the nozzle; and
the end-piece is attached by force-fitting the nozzle inside the tube.

23. The cannula of claim 14, wherein the external diameter of the end-piece is substantially equal to the diameter of the tube.

24. The cannula of claim 14, wherein the tube has an arc of circle shape with a radius of between 140 mm and 190 mm, and a length of between 170 mm and 225 mm.

25. The cannula of claim 14, wherein the tube discharge openings are provided on the tube in a plurality of parallel rows with radial openings, in which the axes of the radial openings of one row are coplanar and uniformly distributed over the circumference of the tube.

26. The cannula of claim 14, wherein the tube discharge openings are provided in eight parallel rows, in which each row has four tube discharge openings and extends over a length substantially equal to or greater than one-half of the length of tube.

27. The cannula of claim 14, wherein the tube comprises visual reference markings on the external surface thereof making a user aware of an insertion point of the cannula.

28. A laryngotracheal anesthesia device comprising:
a syringe configured to contain an anesthetic liquid;
a needle configured to pump the anesthetic liquid into the syringe;
a tube having a proximal end configured for connection to the syringe and a distal end configured for insertion into a laryngeal cavity to a trachea of a patient, the tube having a plurality of tube discharge openings; and
an end piece configured for receipt at the distal end of the tube to receive the anesthetic liquid from the tube, the end piece having a discharge opening through which the anesthetic liquid is discharged.

29. The laryngotracheal anesthesia device of claim 28, wherein the end-piece comprises:
a nozzle configured for receipt at the distal end of the tube; and
a sleeve that covers the nozzle, the sleeve having an axial discharge.

30. The laryngotracheal anesthesia device of claim 29, wherein the nozzle comprises:
a first nozzle segment forming an inlet channel with a first predetermined diameter and a first predetermined length;
a second nozzle segment forming an intermediate channel with a second predetermined diameter and a second predetermined length; and
a third nozzle segment forming a discharge channel with a third predetermined diameter and a third predetermined length.

31. The laryngotracheal anesthesia device of claim 30, wherein:
the first predetermined diameter is around 1 mm and the first predetermined length is around 4.2 mm;
the second predetermined diameter is around 0.5 mm and the second predetermined length is around 3.3 mm; and
the third predetermined diameter is around 0.1 mm and the third predetermined length is around 0.5 mm.

32. The laryngotracheal anesthesia device of claim 28, wherein the tube comprises visual reference markings on the external surface thereof making a user aware of the an insertion point of the cannula.

33. A cannula for a laryngotracheal anesthesia device, the cannula comprising:
a tube having a proximal end configured for connection to a syringe containing an anesthetic liquid and a distal end configured for insertion into a laryngeal cavity to a trachea of a patient, the tube having a plurality of anesthetic liquid discharge openings provided thereon; and
a nozzle configured for receipt at the distal end of the tube and which receives the anesthetic liquid therefrom and through which the anesthetic liquid is discharged, the a nozzle having a first nozzle segment forming an inlet channel with a first predetermined diameter and a first predetermined length, a second nozzle segment forming an intermediate channel which is coaxial with the inlet channel with a second predetermined diameter less than the first predetermined diameter, and a third nozzle segment forming a discharge channel which is coaxial with the intermediate channel with a third predetermined diameter which is less than the second predetermined diameter.
